(19) United States
(12) Patent Application Publication
(10) Pub. No.: US 2026/0174930 A1
(43) Pub. Date: Jun. 25, 2026

(54) MULTILAYERED SEAFOOD-EMULATING CONSUMABLE

(71) Applicants: STEAKHOLDER FOODS LTD., Rehovot (IL); THE IP LAW FIRM OF GUY LEVI, LLC, Wyckoff, NJ (US)

(72) Inventors: Nadav Noor, Tel Aviv (IL); Dan Koziovski, Rehovot (IL); Orit Goldman, Jerusalem (IL)

(21) Appl. No.: 19/129,012

(22) PCT Filed: Nov. 13, 2023
(86) PCT No.: PCT/US2023/037199

§ 371 Date: May 11, 2025

Related U.S. Application Data
(60) Provisional application No. 63/424,926, filed on Nov. 13, 2022.

(51) Int. Cl. A61L 27/36; A61K 8/81; A61L 27/38; A61L 27/54; B29C 64/112

(52) U.S. Cl. CPC A61L 27/3604; A61K 8/8164; A61L 27/383; A61L 27/54; B29C 64/112; A23V 2002/00

(57) ABSTRACT

The disclosure relates to methods, and compositions for a seafood-emulating consumable. More specifically, the disclosure relates to methods and compositions for forming a food consumable emulating seafood structure, in particular, flaky texture.

### BACKGROUND

The disclosure is directed to methods, and compositions for a seafood-emulating consumable. More specifically, the disclosure is directed to methods and compositions for forming a food consumable emulating seafood structure, in particular, flaky texture.

Proteins are important dietary nutrients. They serve as a fuel source or as sources of amino acids, including the essential amino acids that cannot be synthesized by the body, as well as to build muscle mass and improve performance.

Efforts to develop seafood analogs include producing such analogs from vegetable protein and/or seafood sources using extrusion-expansion techniques or other conventional means. These products are in the form of a uniform, homogeneous mass, but lack the typical organoleptic characteristics of real seafood chunks. Therefore, these products are not suitable for use in applications in which the use of simulated seafood chunks is desired. Additional show improvement by processing a seafood emulsion under conditions that produce a layered, non-expanded product in the form of chunks or pieces that simulate real seafood chunks in texture, appearance, and consistency. The product is in the form of distinct chunks or pieces having a plurality of juxtaposed, manually separable seafood-like layers resembling a fibrous chunk of real seafood in appearance, texture, and consistency. However, while these methods produced somewhat acceptable seafood analogs that had the fibrous appearance of pork, beef, and the like, the methods failed to provide fish analogs with an acceptable appearance and texture that mimicked real fish.

Accordingly, there is a need for reliable and commercially practicable methods of forming seafood-emulating consumables.

### SUMMARY

Disclosed, in various implementations, are methods and compositions for forming a food consumable emulating flaky sea-food seafood, using stacked, individually cross-linked layers.

In an exemplary implementation provided herein is method of forming an edible sea-food flesh-emulating product, comprising: using a first composition, forming a plurality of layers, wherein each layer of the plurality of layers is sized, adapted and configured, when assembled-to form the edible sea-food flesh-emulating product or a portion thereof; using a crosslinking composition, crosslinking each layer separately; and assembling the plurality of layers to form the edible sea-food flesh-emulating product.

In another exemplary implementation, the methods of forming an edible sea-food flesh-emulating product, is implemented in a system comprising a drop-on-demand bio-printer, the bio-printer configured to: using a first bio-ink comprising the first composition, print each layer in the plurality of layers, thereby forming the plurality of layers; using a second bio-ink comprising the crosslinking composition, selectively dispensing the second bio-ink onto the plurality of formed layers, wherein the bio-printer comprises: a first bio ink circulating manifold, and a second bio ink circulating manifold, the first bio-ink manifold being in liquid communication with a reservoir storing the first bio ink, and the second bio-ink manifold being in liquid communication with a reservoir storing the second bio ink; a first plurality of dispensing elements arranged in an array being in liquid communication with the first bio-ink manifold, the first plurality of dispensing elements configured to dispense the first bio-ink; a second plurality of dispensing elements arranged in an array in liquid communication with the second bio-ink manifold, the second plurality of dispensing elements configured to dispense the second bio-ink; a central processing module (CPM) comprising at least one processor in communication with the first bio ink circulating manifold, the second bio ink circulating manifold, the first plurality of dispensing elements, and the second plurality of dispensing elements, the CPM being in further communication with a non-transitory memory device, storing thereon a computer-readable media with a set of executable instructions configured when executed by the at least one processor, to: receive a three-dimensional (3D) visualization file of the edible sea-food flesh-emulating product; create a library of layer files, each layer file comprises: at least one substantially two dimensional (2D) pattern to be printed using the first bio-ink, and at least one substantially two dimensional (2D) pattern to be printed using the second bio-ink; using the first plurality of dispensing elements, dispense the first bio-ink; and using the second plurality of dispensing elements, dispense the second bio ink.

### BRIEF DESCRIPTION OF THE FIGURES

For a better understanding of the method and compositions for forming an edible sea-food flesh-emulating product, with regard to the implementations thereof, reference is made to the accompanying images and figures, in which:

FIG. 1 is an image showing the flaky nature of the fish-emulating flesh;

FIG. 2, is an image depicting the flaky fish-emulating consumable before pan cooking;

FIG. 3, is an image depicting the formed flaky fish-emulating consumable after pan frying; and

FIG. 4, is a schematic flow chart illustrating an exemplary implementation of the method disclosed.

### DETAILED DESCRIPTION

While various exemplary implementations are shown and described herein, it will be obvious to those skilled in the art that such exemplary implementations are provided by way of example only. Numerous variations, changes, and substitutions may occur to those skilled in the art without departing from the core proposed technology. It should be understood that various alternatives to the exemplary implementations described herein may be employed.

Provided herein are implementations of methods and compositions for forming flaky fish-emulating consumable, using stacked layers of various compositions capable of forming bio-inks. In the context of the disclosure, the term “Flake” means a thin piece, layer, or small fragment of something, while the term “flaky” means a textural attribute of the fish (and other seafood), naturally caused since connective tissues in fish and other seafood are transformed to gelatin at a much lower temperature than the connective tissues in beef.

The flaky textural attribute is the sensory interpretation and expression of the structure or interior construction of the products and is linked to the response to stress and haptic attributes measured and presented as some mechanical, visual and haptic properties, manifesting as performance of hardness/firmness, gumminess, resilience, cohesiveness, springiness, adhesiveness, and viscosity by the vision, hearing, somesthesis, and kinesthesis of human sense in the muscles of the hand, finger, tongue, jaw, or lips.

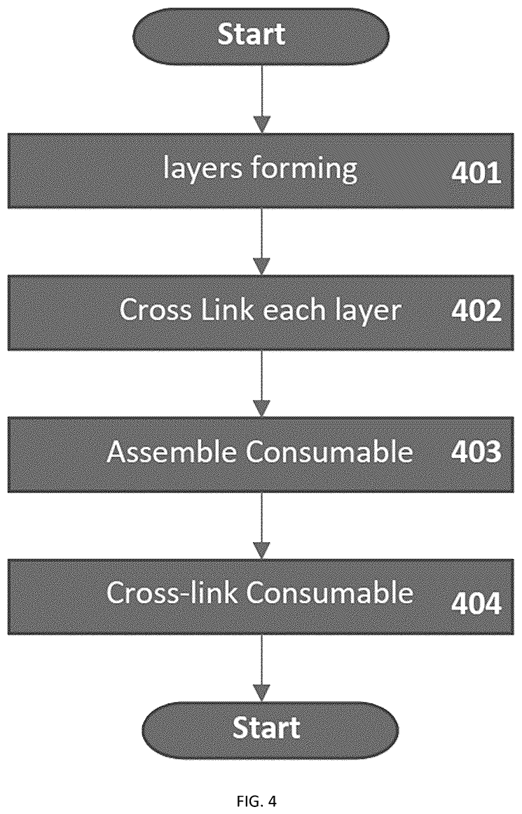

Accordingly, and in an exemplary implementation (illustrated schematically by the flow chart in FIG. 4), provided herein is a method of forming an edible sea-food flesh-emulating product or consumable, comprising: using a first composition, forming a plurality of layers 401, wherein each layer of the plurality of layers is sized, adapted and configured, when assembled-to form the edible sea-food flesh-emulating product or a portion thereof (see e.g., FIG. 2); using a crosslinking composition, crosslinking each layer separately 402; and assembling 403 the plurality of layers to form the edible sea-food flesh-emulating product. As used herein, the term “assembling”, refers to positioning the various cross-linked layers in a shape configured to create the final sea-food consumable.

In the context of the disclosure, the term “consumable” refers to a specific seafood-emulating foodstuff, that is solid, semi-solid and liquid ingestible materials for man or animals which can be any material that provides nourishment for the growth or metabolism of a living organism. Accordingly, the consumable can be in a shape of any cut of seafood, for example, a cross section of a flaky fish filet. Once assembled and treated, the consumable can be further processed to obtain the final form desired. Furthermore, the term “seafood-emulating” means muscle and fat tissue, and/or its'equivalent that has undergone a biochemical process resulting in a composition as well as other physico-chemical and organoleptic characteristics that is substantially equivalent to that of naturally existing and processed by a specific source animal, such as, for example; piscine family members, as well as shrimps, scallops, mollusks and the like.

In an exemplary implementation, each layer of the plurality of layers sized, adapted and configured, when assembled-to form the edible, or consumable sea-food flesh-emulating product or a portion thereof has a predetermined thickness. Each layer can have the same or different layer thickness, (see e.g., FIGS. 2, 3).

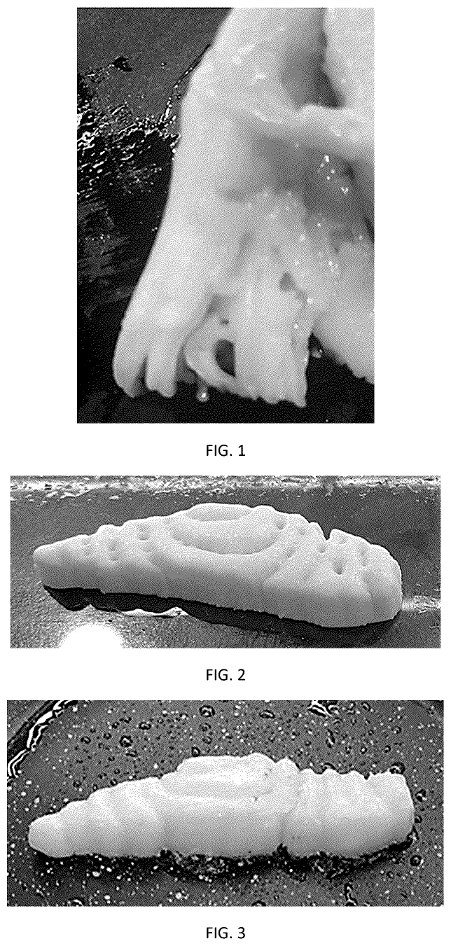

Furthermore, in certain exemplary implementations, following the step of assembling, a step of crosslinking the edible sea-food flesh-emulating product is carried out on the whole product, to achieve final desired texture. In addition, the first composition may further comprise one or more colorants; for example, titanium dioxide, iron oxides; annatto, cochineal, paprika, turmeric, carotenoids, and combinations thereof; wherein the titanium dioxide comprises from 0.1% (w/w) to 2.0% (w/w) of the final product before cooking (see e.g., FIG. 1 image).

The first composition, used to form the plurality of layers, can have a composition that comprises a surfactant (emulsifier) at a concentration of between about 0.01 and about 0.03% (w component/w pre-cooked product); a first and second hydrocolloid, which can be a biocompatible hydrogel-forming polymer, the first hydrocolloid having a concentration of between about 2.0 % and about 3.0% (w/w); a second hydrocolloid having a concentration of between about 0.01% and about 0.07% (w/w); a protein at a concentration of between about 1.0% and about 2.0% (w/w); and water at a concentration of between about 94.9% and about 96.98% (w/w), completing to 100%, in other words, upon determining the final weight of the edible seafood emulating product, and weighing the ingredients, water is added such that the individual ingredients are maintained within the ranges defined. In certain other implementations, the final seafood emulating product further comprises from about 3% (w/w) to about 9% (w/w) fat.

In addition, the first composition, the second composition, or both, can further comprise muscle tissue generated from fish stem cells, or the stem cells of other seafood. The immortalized stem cell lines used can be at least on of: the snakehead murrel, the Sahul Indian catla, the helicopter catfish, and a goldfish cell line.

The first biocompatible hydrogel-forming polymer can be a composition comprising at least one of: an alginate, a carrageenan, an agar, a guar gum, a copolymer, and a terpolymer of the foregoing, while the second biocompatible hydrogel-forming polymer is a composition comprising at least one of: a gelatin, a collagen, an elastin, Poly(lysine-g-(lactide-b-ethylene glycol)), hydroxylated poly(lysine), a xanthan, a copolymer thereof, and a terpolymer thereof.

In the context of the disclosure, the term “copolymer” refers to polymers formed by the polymerization of at least two different monomers. For example, the term “copolymer” includes the co-polymerization reaction product of amines and carbohydrates or the copolymerization of PVP to chitosan. Likewise, the term “terpolymer” refers to polymers formed by the polymerization of at least three different monomers.

In certain exemplary implementations, the surfactant is selected from the group of cationic, zwitterionic, amphoteric and ampholytic surfactants, such as sodium methyl cocoyl taurate, sodium methyl oleoyl taurate, sodium lauryl sulfate, triethanolamine lauryl sulfate and betaines. Likewise, in certain exemplary implementations the surfactant can be one or more of the following: a combination of steareth-2 and steareth-21 on their own or in combination with glyceryl monostearate (GMS); in certain other embodiments the surfactant is a combination of polysorbate 80 and PEG-40 stearate. Additionally, or alternatively, the surfactant is a combination of glyceryl monostearate/PEG 100 stearate. Additionally, or alternatively, the surfactant is a combination of two or more of stearate 21, PEG 40 stearate, and polysorbate 80. Additionally, or alternatively, the surfactant is a combination of two or more of laureth 4, span80, and polysorbate 80. Additionally, or alternatively, the surfactant is a combination of two or more of GMS and ceteareth. Additionally, or alternatively, the surfactant is a combination of two or more of steareth 21, ceteareth 20, ceteth 2 and laureth 4. Additionally, or alternatively, the surfactant is a combination of ceteareth 20 and polysorbate 40 stearate. Additionally, or alternatively, the surfactant is a combination of span 60 and GMS. Additionally, or alternatively, the surfactant is a combination of two or all of PEG 40 stearate, sorbitan stearate and polysorbate 60.

In an exemplary implementation, the first hydrocolloid is an alginate, the second hydrocolloid is a xanthan gum, and the surfactant is span-80.

In addition, the protein can be any suitable protein useful to produce the seafood emulating product (consumable). For example, the protein can be: fish protein, soy protein, soy protein concentrate, soy flour, milk protein, whey protein, plasma protein, egg protein, wheat protein, pea protein, textured vegetable protein (TVP), hydrolyzed vegetable protein (HVP), seitan, or a protein composition comprising one or more of the foregoing.

Likewise, the fat is any suitable fat or oil useful to produce the seafood emulating product (consumable). For example, the fat can be any non-hydrogenated fat or oil of animal or vegetable origin. In various exemplary implementations, the fat is one or more polyunsaturated fatty acids, and/or fish oil. In various embodiments, the fats are monounsaturated fats, polyunsaturated fats, or combinations thereof. In a preferred embodiment, the fat is fish oil.

In an exemplary implementation, the crosslinking composition comprises: a calcium ion at a concentration of between about 0.5% and about 5.0% (w/w); a transglutaminase at a concentration of between about 1.0% to about 3.0% (w/w); water at a concentration of between about 92.0% and about 98.5% (w/w), completing to 100%. The term “crosslinking composition” refers in an exemplary implementation to a composition operable to fuse the layers following assembly. For example, the crosslinking composition can be a composition comprising an enzyme configured to form isopeptide crosslinks, such as for example Tyrosinase, Laccase, and transglutaminase. Additionally, the crosslinking composition can comprise ions operable to crosslink the hydrocolloid of the first composition (e.g., alginate). Additionally, or alternatively, the crosslinking composition can further comprise tannic acid. For example, in certain exemplary implementations, the first hydrocolloid is collagen and the second hydrocolloid is beta-glucan, and the crosslinking composition comprises tannic acid (a form of tannins that are natural phenolic compounds that can form complexes with polysaccharides and proteins). In an exemplary implementation, the tannic acid is used at a concentration of between about 0.1% and about 5.0%, whereby the tannic acid optionally replaces the crosslinking enzyme (e.g., transglutaminase).

In an exemplary implementation the consumables disclosed herein are formed using the methods disclosed herein, is implemented in the systems disclosed. Accordingly, provided herein is a method of forming an edible sea-food flesh-emulating product, implemented in a system comprising a drop-on-demand bio-printer, the bio-printer configured to: using a first bio-ink comprising the first composition, print each layer in the plurality of layers, thereby forming the plurality of layers; using a second bio-ink comprising the crosslinking composition, selectively dispensing the second bio-ink onto the plurality of formed layers comprising: using a first composition, forming a plurality of layers, wherein each layer of the plurality of layers is sized, adapted and configured, when assembled-to form the edible sea-food flesh-emulating product or a portion thereof; using a crosslinking composition, crosslinking each layer separately; and assembling the plurality of layers to form the edible sea-food flesh-emulating product.

The bio printer is, in an exemplary implementation a self-contained unit that comprises substantially all components required to generate the drop-on-demand of the bio-ink composition(s). Generally “bioprinting” the seafood (flesh) emulating product (consumable), refers to a process of making specific type or several types of native or manipulated components, for example, cells or hydrocolloids configured to form the edible tissue analog by depositing material mixed with other bio-inks using inkjet printer having drop-on-demand capabilities. Likewise, the term “forming” (and its variants “formed”, etc.) refers in an exemplary implementation to pumping, injecting, pouring, releasing, displacing, spotting, circulating, nebulizing, spaying, ink-jetting, jetting, or otherwise placing a fluid or material (e.g., the first bio-ink) in contact with another material (e.g., substrate, or another layer) using any suitable inkjet printing method. In an exemplary implementation, “forming” refers to the assembly of the 3D biostructure itself from its underlying 2D layer images, which, in another exemplary implementation are derived from various raster images (e.g., *.dcm) and/or vector data models.

Methods of bioprinting or forming the 3D, seafood flesh-emulating product (consumable) (e.g., tissue, connective tissue) can dispense (and derivatives thereof; are to be understood to refer to any device or technique that deposits, dispenses, transfers or creates material on a surface in a controlled accretive or additive manner) can deposit the first bio-ink, and can be configured to provide the bio-ink droplet(s) upon demand, in other words, as a function of various process parameters such as a conveyor speed, desired layer thickness, layer type, printing speed, and the like.

The bio-printer used to implement the methods disclosed comprises in an exemplary implementation: a first bio ink circulating manifold, and a second bio ink circulating manifold, the first bio-ink manifold being in liquid communication with a reservoir storing the first bio ink, and the second bio-ink manifold being in liquid communication with a reservoir storing the second bio ink; a first plurality of dispensing elements arranged in an array being in liquid communication with the first bio-ink manifold, the first plurality of dispensing elements configured to dispense the first bio-ink; a second plurality of dispensing elements arranged in an array in liquid communication with the second bio-ink manifold, the second plurality of dispensing elements configured to dispense the second bio-ink; a central processing module (CPM) comprising at least one processor in communication with the first bio ink circulating manifold, the second bio ink circulating manifold, the first plurality of dispensing elements, and the second plurality of dispensing elements, the CPM being in further communication with a non-transitory memory device, storing thereon a computer-readable media with a set of executable instructions configured when executed by the at least one processor, to: receive a three-dimensional (3D) visualization file of the edible sea-food flesh-emulating product; create a library of layer files, each layer file comprises: at least one substantially two dimensional (2D) pattern to be printed using the first bio-ink, and at least one substantially two dimensional (2D) pattern to be printed using the second bio-ink; using the first plurality of dispensing elements, dispense the first bio-ink; and using the second plurality of dispensing elements, dispense the second bio ink.

In an exemplary implementation, the CPM is operable to generate, for each file representing the substantially 2D layer of the first bio-ink composition, to further generate a sub library of crosslinking composition pattern files, each crosslinking composition pattern file representing a substantially 2D layer for printing the crosslinking composition. Each of the sub library files of crosslinking composition patterns, can further comprise a metafile with at least one of: an order of printing, an identifier of the file of substantially 2D layer of the first bio-ink composition it is associated with, and instructions on at least one of: speed of printing, amount of material printed and printing order. In addition, the crosslinking composition pattern in each file can be identical, or at least one file can have a different pattern than another file in the sub library.

In addition, the computer program, can comprise program code means for carrying out the steps of the methods described herein, as well as a computer program product comprising program code means stored on a medium that can be read by a computer. Non-transitory storage device(s) as used in the methods described herein can be any of various types of non-volatile memory devices or storage devices (in other words, memory devices that do not lose the information thereon in the absence of power). The term “memory device” is intended to encompass an installation medium, e.g., a CD-ROM, floppy disks, or tape device or a non-volatile memory such as a magnetic media, e.g., a hard drive, optical storage, or ROM, EPROM, FLASH, etc. The memory device may comprise other types of memory as well, or combinations thereof. In addition, the memory medium may be located in a first computer in which the programs are executed (e.g., the 3D inkjet printer provided), and/or may be located in a second different computer which connects to the first computer over a network, such as the Internet. In the latter instance, the second computer may further provide program instructions to the first computer for execution. The term “memory device” can also include two or more memory devices which may reside in different locations, e.g., in different computers that are connected over a network. Accordingly, for example, the bitmap library can reside on a memory device that is remote from the CAM module coupled to the bio-printer provided, and be accessible by the 3D bio-printer provided (for example, by a wide area network).

### EXAMPLE I

1. Print one layer of alginate-containing bio ink;
2. Crosslink using spraying of calcium and transglutaminase (TG) solution;
3. Print successive layers and repeat crosslinking until the 3D model is complete;
4. Incubate for 2 hours at a temperature between about 24° C. and about 37° C. degrees for the TG to crosslink the proteins in the bio-ink;
5. Incubate for 24 hours at 4° C. for dehydration of the 3d Structured consumable;
6. Incubate in oven at between about 140° C. and about 180° C. for between about 5 minutes and about 15 minutes to inactivate enzymes and kill incidental microorganisms; and
7. Package.

In the context of the disclosure, the term “operable” means a certain component, compound, element or step, the system and/or the device and/or the program, is sized, adapted and calibrated, fully functional, comprises elements for, chemically and/or biologically reactive, and meets applicable operability requirements to perform a recited function when disposed, activated, coupled, implemented, actuated, effected, realized, or when an executable program is executed by at least one processor associated with the system and/or the device. In relation to systems and circuits, the term “operable”, as used herein, means the system and/or the circuit is fully functional and calibrated, comprises logic for, having the hardware and firmware necessary, as well as the circuitry for, and meets applicable operability requirements to perform a recited function when executed by at least one processor.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

All ranges disclosed herein are inclusive of the endpoints, and the endpoints are independently combinable with each other. “Combination” is inclusive of blends, mixtures, alloys, reaction products, and the like. The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the cell(s) includes one or more cell). Reference throughout the specification to “one implementation”, “another implementation”, “an implementation”, “an exemplary implementation” and so forth, when present, means that a particular element (e.g., feature, structure, and/or characteristic) described in connection with the implementation is included in at least one implementation described herein, and may or may not be present in other implementations. In addition, it is to be understood that the described elements may be combined in any suitable manner in the various implementations.

Furthermore, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. For example, “about” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

Accordingly and in an exemplary implementation, provided herein is a method of forming an edible sea-food flesh-emulating product, comprising: using a first composition, forming a plurality of layers, wherein each layer of the plurality of layers is sized, adapted and configured, when assembled-to form the edible sea-food flesh-emulating product or a portion thereof; using a crosslinking composition, crosslinking each layer separately; and assembling the plurality of layers to form the edible sea-food flesh-emulating product, wherein (i) each layer has a predetermined thickness, the method further comprising (ii) following the step of assembling, crosslinking the edible sea-food flesh-emulating product, wherein (iii) the first composition comprises: a surfactant at a concentration of between about 0.01 and about 0.03% (w/w); a first hydrocolloid at a concentration of between about 2.0 % and about 3.0% (w/w); a second hydrocolloid at a concentration of between about 0.01% and about 0.07% (w/w); a protein at a concentration of between about 1.0% and about 2.0% (w/w); and water at a concentration of between about 94.9% and about 96.98% (w/w), completing to 100%, wherein (iv) the first hydrocolloid is an alginate, the second hydrocolloid is a xanthan gum, and the surfactant is span-80, (v) the protein is textured vegetable protein (TVP), hydrolyzed vegetable protein (HVP), soy protein, pea protein, seitan, or a protein composition comprising one or more of the foregoing, (vi) the crosslinking composition comprises: a calcium ion at a concentration of between about 0.5% and about 5.0% (w/w); a transglutaminase at a concentration of between about 1.0% to about 3.0% (w/w); water at a concentration of between about 92.0% and about 98.5% (w/w), completing to 100%, wherein (vii) the crosslinking composition further comprising tannic acid at a concentration of between about 0.1% and about 5.0%, the tannic acid optionally replacing the transglutaminase, (viii) implemented in a system comprising a drop-on-demand bio-printer, the bio-printer configured to: using a first bio-ink comprising the first composition, print each layer in the plurality of layers, thereby forming the plurality of layers; using a second bio-ink comprising the crosslinking composition, selectively dispensing the second bio-ink onto the plurality of formed layers, wherein (ix) the bio-printer comprises: a first bio ink circulating manifold, and a second bio ink circulating manifold, the first bio-ink manifold being in liquid communication with a reservoir storing the first bio ink, and the second bio-ink manifold being in liquid communication with a reservoir storing the second bio ink; a first plurality of dispensing elements arranged in an array being in liquid communication with the first bio-ink manifold, the first plurality of dispensing elements configured to dispense the first bio-ink; a second plurality of dispensing elements arranged in an array in liquid communication with the second bio-ink manifold, the second plurality of dispensing elements configured to dispense the second bio-ink; a central processing module (CPM) comprising at least one processor in communication with the first bio ink circulating manifold, the second bio ink circulating manifold, the first plurality of dispensing elements, and the second plurality of dispensing elements, the CPM being in further communication with a non-transitory memory device, storing thereon a computer-readable media with a set of executable instructions configured when executed by the at least one processor, to: receive a three-dimensional (3D) visualization file of the edible sea-food flesh-emulating product; create a library of layer files, each layer file comprises: at least one substantially two dimensional (2D) pattern to be printed using the first bio-ink, and at least one substantially two dimensional (2D) pattern to be printed using the second bio-ink; using the first plurality of dispensing elements, dispense the first bio-ink; and using the second plurality of dispensing elements, dispense the second bio ink, wherein (x) the first hydrocolloid composition, the second hydrocolloid or both further comprises a muscle tissue generated from fish stem cells, (xi) the muscle tissue generated from fish stem cells is generated from immortalized stem cell lines of at least on of: a snakehead murrel, a Sahul Indian catla, a helicopter catfish, and a goldfish immortalized cell line, wherein (xii) the first hydrocolloid is formed by a biocompatible hydrogel-forming polymer composition, (xiii) the first biocompatible hydrogel-forming polymer can be a composition comprising at least one of: an alginate, a carrageenan, an agar, a guar gum, a copolymer, and a terpolymer of the foregoing, wherein (xiv) the second hydrogel composition is formed by a second biocompatible hydrogel forming polymer, and (xv) the second biocompatible hydrogel-forming polymer is a composition comprising at least one of: a gelatin, a collagen, an elastin, Poly(lysine-g-(lactide-b-ethylene glycol)), hydroxylated poly(lysine), a xanthan, a copolymer thereof, and a terpolymer thereof.

In another exemplary implementation, provided herein, is an edible seafood flesh-emulating product formed using the methods disclosed herein.

Although the foregoing disclosure for methods and compositions for forming a food consumable emulating source-specific seafood, using stacked layers, has been described in terms of some implementations, other implementations will be apparent to those of ordinary skill in the art from the disclosure herein. Moreover, the described implementations have been presented by way of example only, and are not intended to limit the scope of the inventions. Indeed, the novel methods, systems and compositions described herein may be embodied in a variety of other forms without departing from the spirit thereof. Accordingly, other combinations, omissions, substitutions and modifications will be apparent to the skilled artisan in view of the disclosure herein.

1. A method of forming an edible sea-food flesh-emulating product, comprising:
a. using a first composition, forming a plurality of layers, wherein each layer of the plurality of layers is sized, adapted and configured, when assembled—to form the edible sea-food flesh-emulating product or a portion thereof, wherein the first composition comprises:
i. a surfactant at a concentration of between about 0.01 and ab out 0.03% (w/w);
ii. a surfactant at a concentration of between about 0.01 and about 0.03% (w/w);
iii. a second hydrocolloid at a concentration of between about 0.01% and about 0.07% (w/w);
iv. a protein at a concentration of between about 1.0% and about 2.0% (w/w); and
v. water at a concentration of between about 94.9% and about 96.98% (w/w), completing to 100%;
b. using a crosslinking composition, crosslinking each layer separately; and
c. assembling the plurality of layers to form the edible sea-food flesh-emulating product.

2. The method of claim 1, wherein each layer has a predetermined thickness.

3. The method of claim 1, further comprising, following the step of assembling, crosslinking the edible sea-food flesh-emulating product.

4. (canceled)

5. The method of claim 1, wherein the first hydrocolloid is an alginate, the second hydrocolloid is a xanthan gum, and the surfactant is span-80.

6. The method of claim 1, wherein the protein is textured vegetable protein (TVP), hydrolyzed vegetable protein (HVP), soy protein, pea protein, seitan, or a protein composition comprising one or more of the foregoing.

7. The method of claim 1, wherein the crosslinking composition comprises:
a. a calcium ion at a concentration of between about 0.5% and about 5.0% (w/w);
b. a transglutaminase at a concentration of between about 1.0% to about 3.0% (w/w);
c. water at a concentration of between about 92.0% and about 98.5% (w/w), completing to 100%.

8. The method of claim 7, wherein the crosslinking composition further comprising tannic acid at a concentration of between about 0.1% and about 5.0%, the tannic acid optionally replacing the transglutaminase.

9. The method of claim 8, implemented in a system comprising a drop-on-demand bio-printer, the bio-printer configured to:
a. using a first bio-ink comprising the first composition, print each layer in the plurality of layers, thereby forming the plurality of layers;
b. using a second bio-ink comprising the crosslinking composition, selectively dispensing the second bio-ink onto the plurality of formed layers.

10. The method of claim 9, wherein the bio-printer comprises:
a. a first bio ink circulating manifold, and a second bio ink circulating manifold, the first bio-ink manifold being in liquid communication with a reservoir storing the first bio ink, and the second bio-ink manifold being in liquid communication with a reservoir storing the second bio ink;
b. a first plurality of dispensing elements arranged in an array being in liquid communication with the first bio-ink manifold, the first plurality of dispensing elements configured to dispense the first bio-ink;
c. a second plurality of dispensing elements arranged in an array in liquid communication with the second bio-ink manifold, the second plurality of dispensing elements configured to dispense the second bio-ink;
d. a central processing module (CPM) comprising at least one processor in communication with the first bio ink circulating manifold, the second bio ink circulating manifold, the first plurality of dispensing elements, and the second plurality of dispensing elements, the CPM being in further communication with a non-transitory memory device, storing thereon a computer-readable media with a set of executable instructions configured when executed by the at least one processor, to:
i. receive a three-dimensional (3D) visualization file of the edible sea-food flesh-emulating product;
ii. create a library of layer files, each layer file comprises: at least one substantially two dimensional (2D) pattern to be printed using the first bio-ink, and at least one substantially two dimensional (2D) pattern to be printed using the second bio-ink;
iii. using the first plurality of dispensing elements, dispense the first bio-ink; and
iv. using the second plurality of dispensing elements, dispense the second bio ink.

11. The method of claim 1, wherein the first hydrocolloid composition, the second hydrocolloid or both further comprises a muscle tissue generated from fish stem cells.

12. The method of claim 12, wherein the muscle tissue generated from fish stem cells is generated from immortalized stem cell lines of at least on of: a snakehead murrel, a Sahul Indian catla, a helicopter catfish, and a goldfish immortalized cell line.

13. The method of claim 1, wherein the first hydrocolloid is formed by a biocompatible hydrogel-forming polymer composition.

14. The method of claim 13, wherein the first biocompatible hydrogel-forming polymer can be a composition comprising at least one of: an alginate, a carrageenan, an agar, a guar gum, a copolymer, and a terpolymer of the foregoing.

15. The method of claim 1, wherein the second hydrogel composition is formed by a second biocompatible hydrogel-forming polymer.

16. The method of claim 15, wherein the second biocompatible hydrogel-forming polymer is a composition comprising at least one of: a gelatin, a collagen, an elastin, Poly(lysine-g-(lactide-b-ethylene glycol)), hydroxylated poly(lysine), a xanthan, a copolymer thereof, and a terpolymer thereof.

17. (canceled)
